(19) United States
(12) Patent Application Publication
(10) Pub. No.: US 2018/0036400 A1
(43) Pub. Date: Feb. 8, 2018

(54) DOUBLE ENGINEERED HIV-1 ENVELOPES

(71) Applicant: DUKE UNIVERSITY, Durham, NC (US)

(72) Inventors: Barton F Haynes, Durham, NC (US); Hua-Xin Liao, Durham, NC (US); S. Munir Alam, Durham, NC (US)

(21) Appl. No.: 15/320,432

(22) PCT Filed: Jun. 25, 2015
(86) PCT No.: PCT/US15/37754

§ 371 Date: Dec. 20, 2016

Related U.S. Application Data
(60) Provisional application No. 62/016,792, filed on Jun. 25, 2014.

(51) Int. Cl. A61K 39/21; C07K 14/005

(52) U.S. Cl. CPC A61K 39/21; C07K 14/005; C12N 2740/16122; C12N 2740/16134; C12N 2740/16071

(57) ABSTRACT

In certain aspects the invention provides HIV-1 engineered envelope proteins and their uses. The engineered envelopes comprise a sequence that prevents cleavage of the envelope associated with recombinant expression in a cell line, and N-terminal deletion which improves envelope expression as a monomer.

This application claims the benefit of U.S. Application Ser. No. 62/016,792 filed Jun. 25, 2014. The content of this application is herein incorporated by reference in its entirety.

### GOVERNMENT INTERESTS

This invention was made with government support under grants AI067854 and AI100645 awarded by the National Institutes of Allergy and infectious Diseases (NIAID, NIH). The government has certain rights in the invention.

### FIELD OF THE INVENTION

The present invention relates in general, to engineered, recombinantly produced HIV-1 envelope and compositions comprising these envelopes, nucleic acids encoding these engineered envelopes, and various methods of use.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

The present invention provides engineered, recombinantly produced HIV-1 envelopes and compositions comprising these envelopes. The invention also provides methods of using these engineered HIV-1 envelopes. In certain embodiments these compositions are suitable for use in inducing anti-HIV-1 antibodies. In particular, provided are immunogenic compositions comprising envelope proteins and/or nucleic acids to induce cross-reactive neutralizing antibodies and increase antibody breadth of coverage. Non-limiting embodiments include methods of inducing broadly neutralizing anti-HIV-1 antibodies using the inventive compositions, in any suitable immunization regimen.

In certain aspects the invention provides an engineered HIV-1 envelope of FIG. 1. In certain aspects the invention provides a double engineered Inv-1 envelope of SEQ lD NO: 2 (B63521 Δ11gp120mutC); SEQ ID NO: 4 (B.6240Δ11gp120mutC): SEQ ID NO: 6(B.9021 gp140CmutC); SEQ ID NO: 8 (B.ADAΔ11gp120mutC) or SEQ ID NO: 10 (JRFLΔ11gp120mutC). In certain embodiments, the envelope is recombinantly produced in any suitable cells line, including but limited to CHO cells. In certain embodiments, the envelope is a monomer.

In certain aspects the invention provides a nucleic acid comprising a sequence encoding an engineered HIV-1 envelope of FIG. 1. A nucleic acid comprising a sequence encoding the envelope of SEQ ID NO: 2, 4, 6, 8 or 10. In certain embodiments, the nucleic acid is of SEQ NO: 1, 3, 5, 7, or 9.

In certain aspects the invention provides a composition comprising the double engineered envelope of the invention. In certain aspect the invention provides a composition comprising a nucleic acid encoding the double engineered envelope of the invention. In certain embodiments, the composition is a pharmaceutical composition comprising any suitable career, excipient, adjuvant and the like.

in certain aspects the invention provides a method of inducing an immune response in a subject comprising administering to the subject a composition comprising, any of the engineered envelopes of the invention, or nucleic acid encoding these, in an amount sufficient to induce an immune response. In certain aspects, the composition is administered as a boost. In certain embodiments these envelopes are suitable for use in inducing anti-HIV-1 antibodies. In certain embodiments these immunogenic compositions comprising envelope proteins and/or nucleic acids are used to induce cross-reactive neutralizing antibodies and increase breadth of coverage. The invention also relates to methods of inducing such broadly neutralizing anti-HIV-1 antibodies using such compositions.

### BRIEF DESCRIPTION OF THE DRAWINGS

FIG. 1 shows nucleic acid and amino acid sequences of double engineered envelopes comprising delta N-terminal deletion and V3 cleavage resistant sequence. The capitalized nucleotides depicted in SEQ ID NOS: 1, 3, 5, 7, and 9 correspond to coding regions, respectively.

FIG. 2 shows Clade B Engineered Env B63521 grown in CHO cells: SEC profile showing monomeric gp120.

FIG. 3 shows Clade B engineered Env B63521 gp120 grown in CHO cells: CD4 binding and CDi epitope upregulation.

### DETAILED DESCRIPTION

In certain aspects the invention provides HIV-1 engineered envelope proteins, or a functional fragment thereof, which comprise a sequence that prevents cleavage of the envelope associated with recombinant expression in cells, e.g. CHO cells, and N-terminal deletion which improves envelope expression as a monomer. In certain embodiments, the N-terminal deletion also improves antigenicity of the engineered envelope. In certain embodiments the present invention provides engineered HIV-1 envelope proteins suitable for a large scale recombinant expression, e.g. but not limited in a CHO cell line. In certain embodiments, the double engineered proteins are purified and are suitable for use in in vitro and in vivo studies, including clinical trials.

In certain embodiments HIV envelope designed in accordance with the present invention involves deletion of residues (e.g., 5-11, 5, 6, 7, 8, 9,10or 11 amino acids) at the N-terminus. For delta N-terminal design, amino acid residues ranging from 4 residues or even fewer to 14 residues or even more are deleted. These residues are between the maturation (signal peptide, usually ending with CX, X can be any amino acid) and “VPVXXXX . . . ”. In certain embodiments all amino acids between the maturation (signal peptide, usually ending with CX, X can be any amino acid) and “VPVXXXX . . . ” sequence are deleted. In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 11 amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins, for example gp120s, expressed mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

Envelopes were engineered by eliminating cleavage of recombinant HIV-I Envs produced, for example, in DHFR-deficient CHO cells. Most of HIV-1 gp 120 proteins expressed in CHO cells are cleaved, while the same gp1.20 proteins expressed in HEK293 (293F) cells are produced as intact proteins. Similarly, HIV-1 B.63521 gp140 Env proteins are produced as cleaved forms in CHO cells, while the same gp 140 proteins express as intact proteins in HEK293 cells in SDS-PAGE, the cleaved HIV-1 Env proteins produced in CHO cells appear as intact proteins under non-reducing conditions, however, they migrate as ˜75 Kd and ˜50 Kd cleaved proteins bands under reducing conditions. These results suggest that HIV-1 Env gp 120 and gp 140 proteins are produced as cleaved products and appear as intact proteins as a result of disulfide bond formation. See PCT/US2014/032497 published as WO2014165494, specifically Example 1, the content of which application is herein incorporated by reference in its entirety.

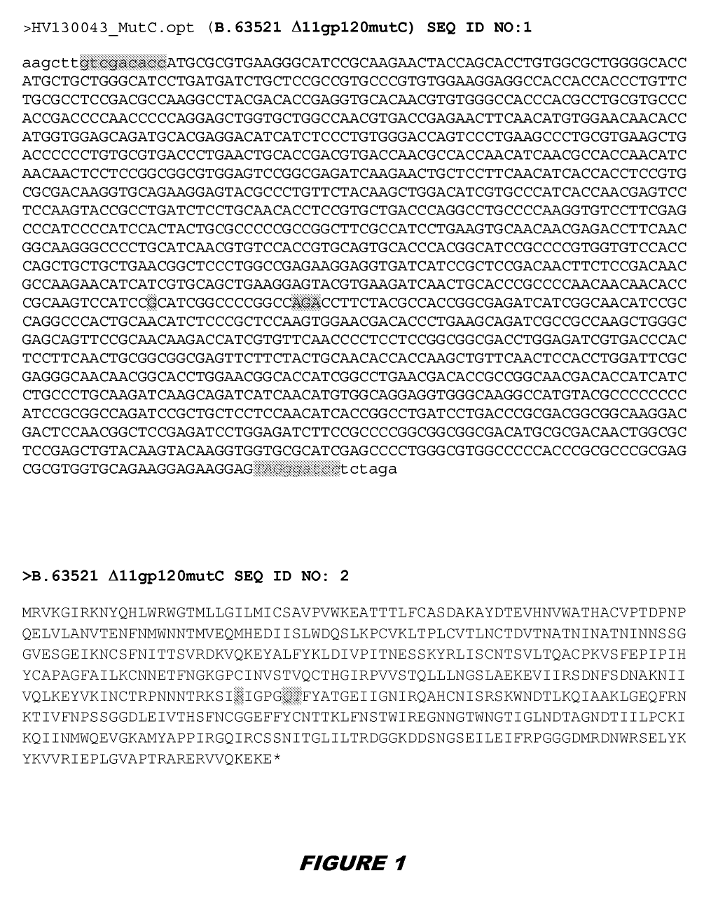

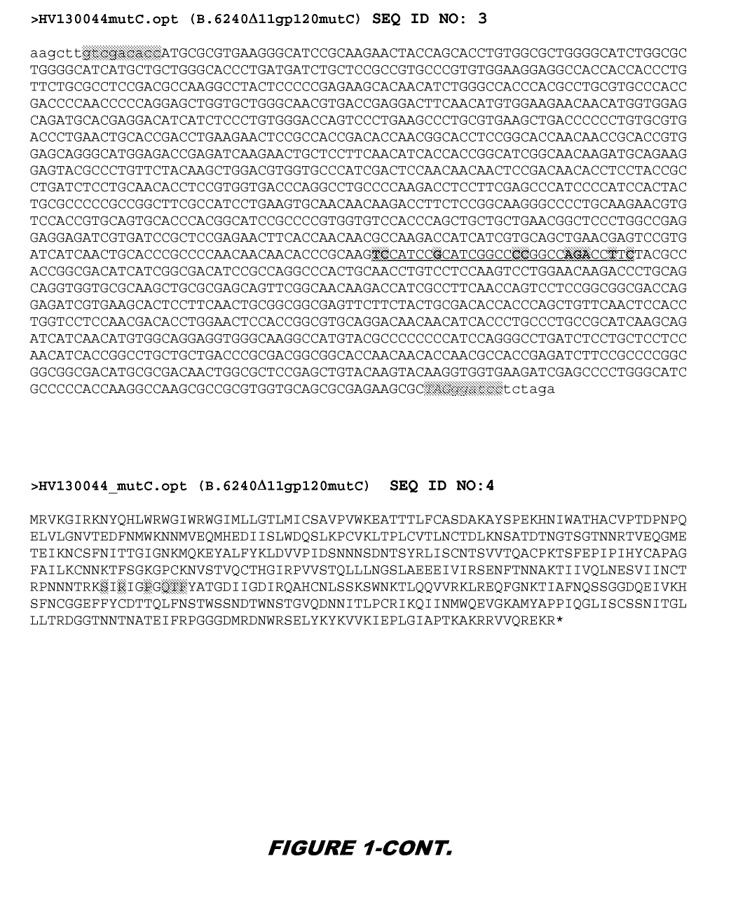

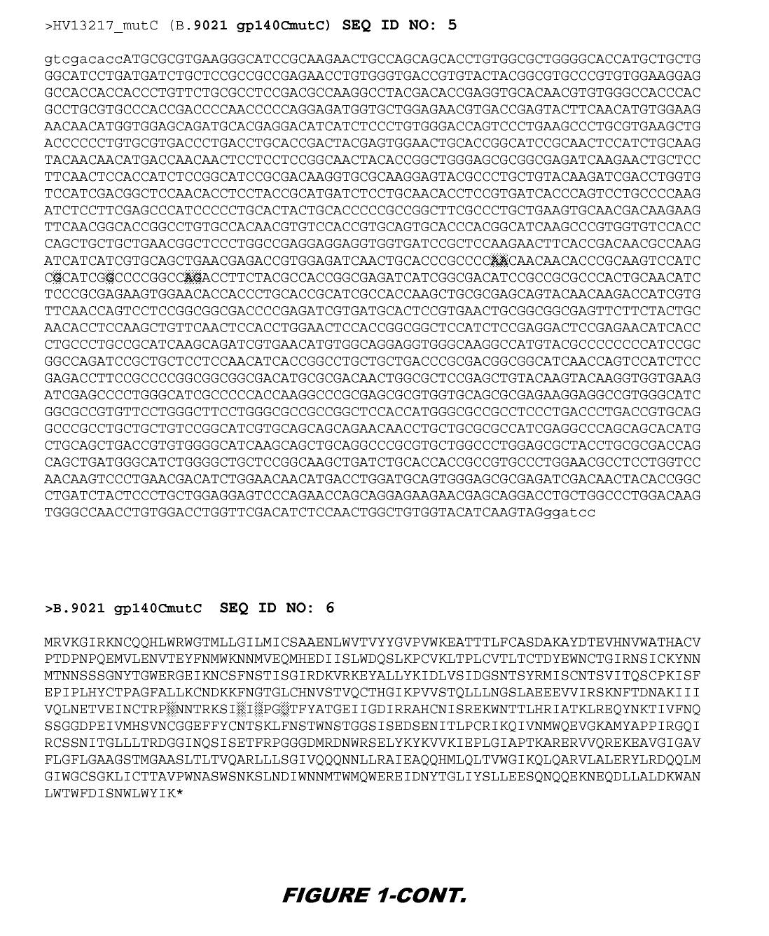

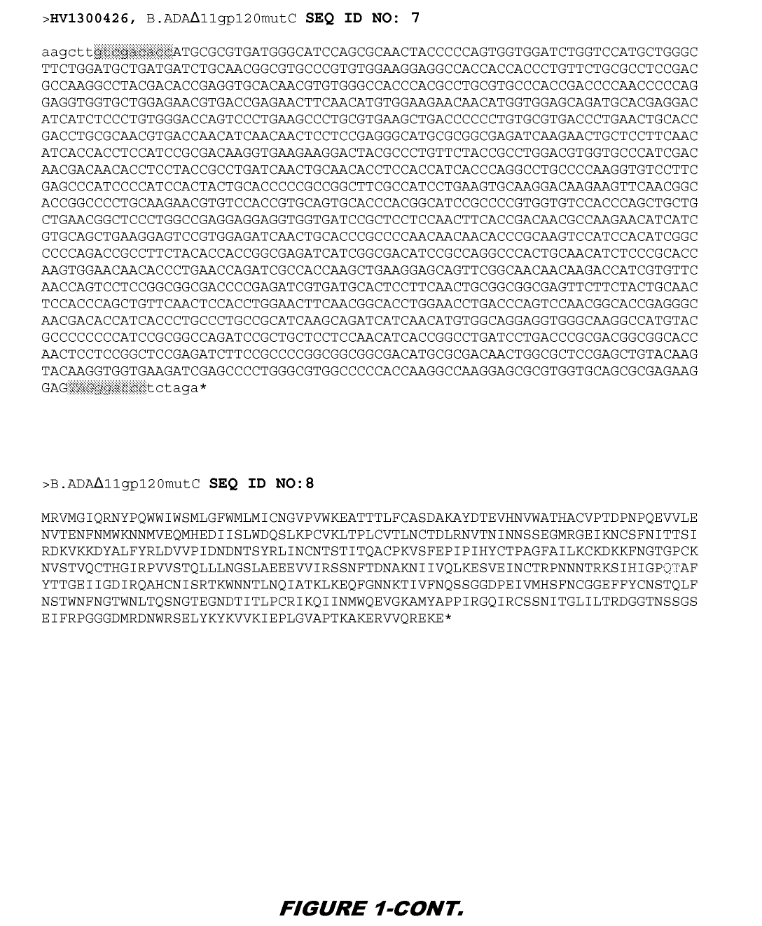

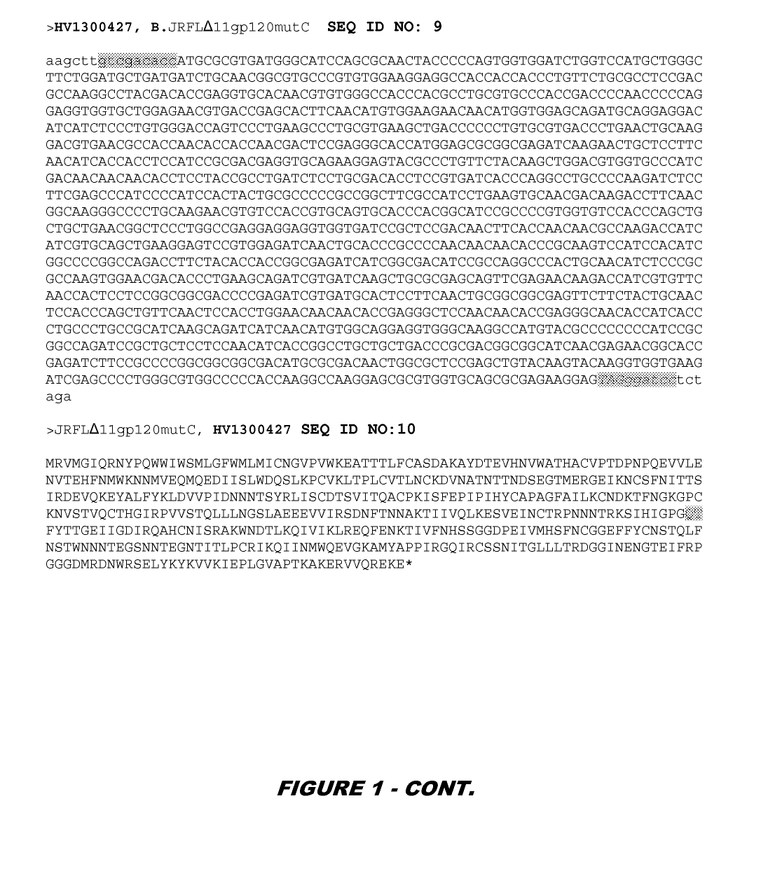

In certain embodiments the V3 loop sequence of the C.1086 env protein (TRPNNNTRKSIRIGPGQTFYATGDIIGNIRQAH) was used to modify HIV-1 envelopes, for example gp 120, ₄)140 or gp160 envelopes, so as to render them resistant to cleavage when produced in CHO cells (referred to as “mutC”, see FIG. 1). In other embodiments, the V3 loop sequence from any clade C envelope can be used to create mutC comprising envelopes.

The properties of the double engineered envelopes of the invention, including but not limited to immunogenicity, antigenicity, solubility, etc. can be characterized in any other suitable assays, including but not limited to assays as described herein.

In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help acid cytotoxic T cell induction. In some embodiments, the mosaic genes any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics, in some embodiments the mosaic genes are trivalent. In some embodiments, the mosaic genes administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the invention contemplates using immunogenic compositions wherein immunogens are delivered as recombinant proteins. Various methods for production and purification of recombinant proteins suitable for use in immunization are known in the art.

The immunogenic envelopes can also be administered as a protein boost in combination with a variety of nucleic acid envelope primes (e.g., HIV-1 Envs delivered as DNA expressed in viral or bacterial vectors).

Nucleotide-based vaccines offer a flexible vector format to immunize against virtually any protein antigen. Currently, two types of genetic vaccination are available for testing DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+T-Cell Responses after rAd5 Boost in a Randomized Clinical Trial, PLoS ONE 8(4): e59340, page 9. Various technologies for delivery of nucleic acids, as DNA and/or RNA, so as to elicit one response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (Barouch D H, et al. Nature Med. 16: 319-23, 2010), recombinant mycobacteria (i.e., rBCG or M smegmatis) (Yu J S et al. Clinical Vaccine; Immunol. 14: 886-093,2007; ibid 13: 1204-11,2006), and recombinant vaccinia type of vectors (Santra S. Nature Med. 16: 324-8, 2010), for example but not limited to ALVAC, replicating (Kibler K V et al., PLoS One 6: e25674, 2011 Nov. 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer amphiphilic block copolymer 704), See Cany et al., Journal of Hepatology 2011 vol. 54 j 115-121; Amaoty et al., Chapter 17 in Yves Bigot (ed.), Mobile Genetic Elements: Protocols and Genomic Applications, Methods in Molecular Biology, vol., 859, pp 293-305 (2012); Arnaoty et al. (2013) Mol Genet Genomics, 2013August; 288(7-8):347-63. Nanocarrier technologies called Nanotaxi® for immunogenic macromolecules (DNA, RNA, Protein) delivery are under development. See for example technologies developed by In-cellart.

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramascular (IM), via subcutaneous, via intravenous, via nasal, via mucosal routes.

The compositions can be formulated with appropriate carriers and adjuvants using techniques to yield compositions suitable for immunization. The compositions can include an adjuvant, such as,for example but not limited to, alum, poly IC, MF-59 or other squalene-based adjuvant, ASOIB, or other liposomal based adjuvant suitable for protein or nucleic acid immunization. In certain embodiments, TLR agonists are used as adjuvants. In other embodiment, adjuvants which break immune tolerance are included in the immunogenic compositions.

There are various host mechanisms that control bNAbs. For example highly somatically mutated antibodies become autoreactive and/or less fit (immunity 8: 751, 1998; PloS Comp. Biol. 6 e1000800 , 2010; J. Thoret. Biol. 164:37, 1993); Polyreactive/autoreactive naïve B cell receptors (unmutated common ancestors of clonal lineages) can lead to deletion of Ab precursors (Nature 373: 252, 1995; PNAS 107: 181, 2010; J. Immunol. 187: 3785, 2011); Abs with long HCDR3 can be limited by tolerance deletion (JI 162: 6060, 1999; JCI 108: 879, 2001). BnAb knock-in mouse models are providing insights into the various mechanisms of tolerance control of MPER BnAb induction (deletion, anergy, receptor editing). Other variations of tolerance control likely will be operative in limiting BnAbs with long HCDR3s, high levels of somatic hypermutations. The compositions and methods of the invention can he used in combination with any agent and method to reducing the effects of host tolerance controls in the production of HIV-1 bnAbs.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

As will be apparent to one of ordinary skill in the art from a reading of this disclosure, the embodiments of the present disclosure can be embodied in forms other than those specifically disclosed above. The particular embodiments described herein are, therefore, to be considered as illustrative and not restrictive. Those skilled in the art will recognize, or be able to ascertain, using no more than routine experimentation, numerous equivalents to the specific embodiments described herein. The scope of the invention is as set forth in the appended claims and equivalents thereof, rather than being limited to the examples contained in the foregoing description.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

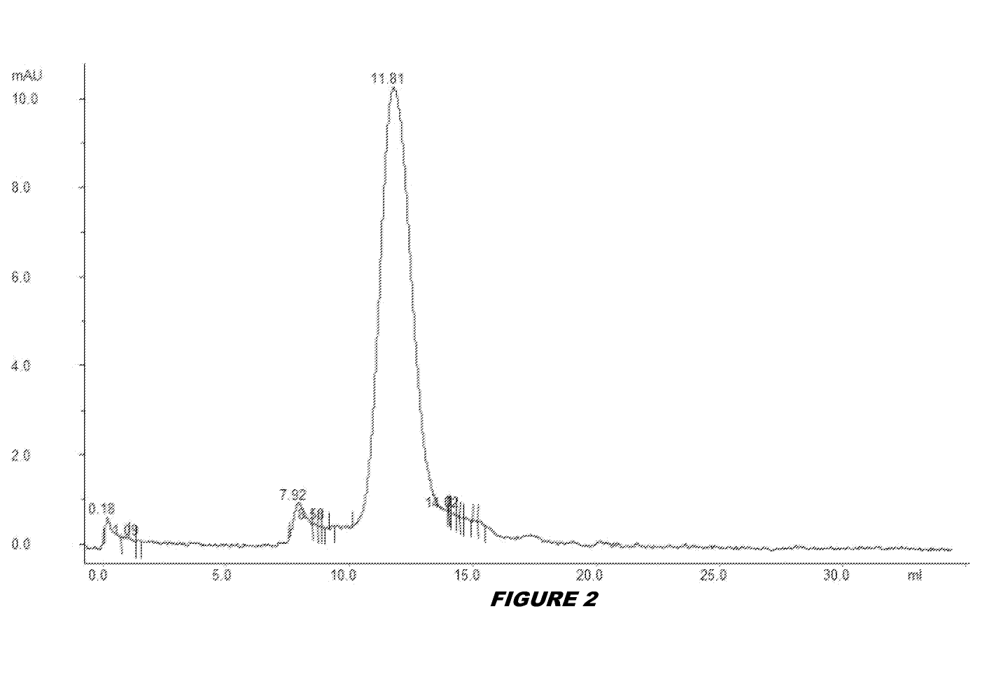

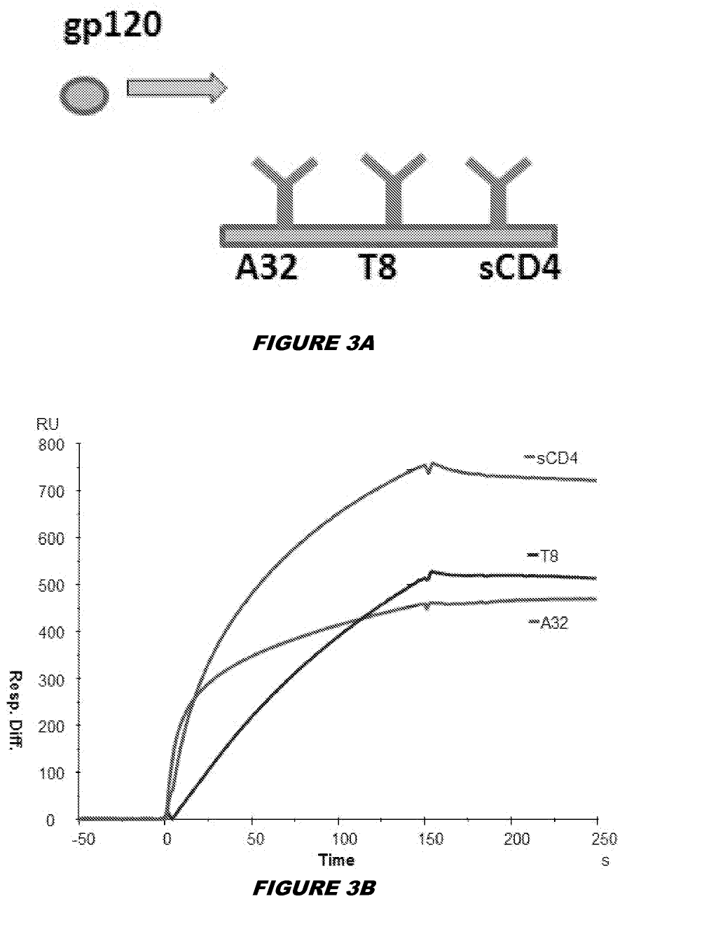

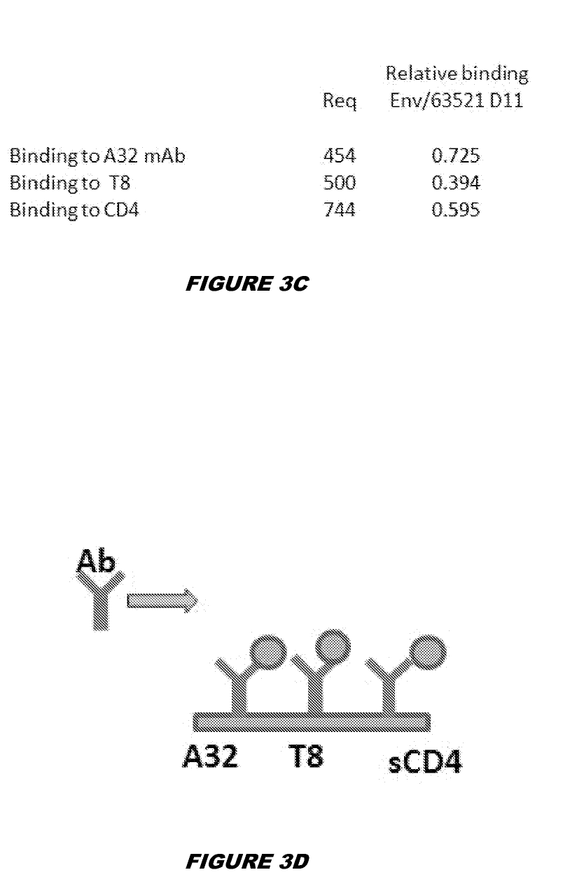

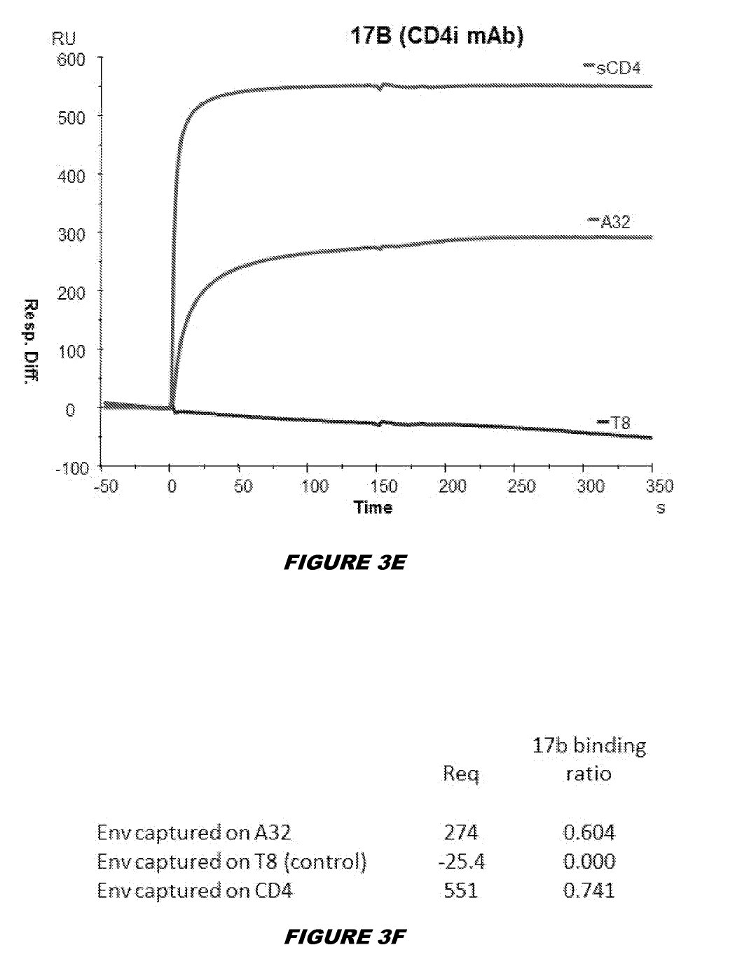

Properties of the double engineered B63521 envelope were determined in various assays. FIG. 2 shows that the envelope is expressed as a monomer. FIG. 2 shows chromatography profile of a CHO expressed and purified protein. The antigenicity of double engineered gp120 envelope B63251 was determined in an antibody binding assay. FIG. 3 shows that the double engineered gp120 envelope B63251 is expressed as a monomer and retains its properties, as demonstrated by its binding to 17B, which is a CD4 binding site antibody.

### Example 2

Comparing Bivalent (Clade B/E) and Pentavalent Boost (B/E/E/E/E) in Non-Human Primates

This example studies envelopes of the invention in combination with the original RV144 vaccine ((Berks-Ngarm et al, N, Eng, J. Med. 361: 2209-20 (2009)) to improve the coverage by a new vaccine formulation of the epitope diversity in the V2 region.

In certain embodiments, the invention provide an immunization regimen with ALVAC-HIV vPC1521 prime X2 then ALVAX vPC1521 boost X2 with A244 gp 120 Delta 11 +B.63521 Delta 11gp120+AA104.0 delta 11 or 7 gp120 +AA107.0 delta 11 or 7gp 120+AA058.1 delta 11 or 7 gp120. An alternate set of AA Envs is AA072.1, AA009.1. and AA015.1, See WO 2014/17235 at FIGS. 1, 5, 6.

In certain embodiments, the gp120 envelopes are double engineered to include deltaN deletion and mutC change as described herein, for example in FIG. 1. AA Envs which are deltaN mutC envelopes can be engineered from the sequences in WO 2014/17235 at FIGS. 1, 5, 6.

Group A (bivalent boost)-ALVAC vPC1521 prime X2, then ALVAC VPC1521+B/E boost X2 (B.6240 gp120D11+A244 gp120 D11 in GLA/SE), or optionally

Group B Group 4 (bivalent boost)-ALVAC vPC1521 prime X2, then ALVAC VPC1521+B/E boost X2 (B.63521 gp120D11+A244 gp120 D11 in GLA/SE)

Group C (pentavalent boost)-ALVAC vPC1521 prime X2, then ALVAC VPC1521+B/E boost X2 (B.6240 gp120D11 +A244 gp120 D11+new three valent AE gp120s in GLA/SE)—new trivalent gp120s include: AA104.0 delta 11 or 7 gp120+AA107.0 delta11 or 7gp 120 +AA058.1 delta 11 or 7 gp120.

Group D (pentavalent boost)-ALVAC vPC1521 prime X2, then ALVAC VPC1521+B/E boost X2 (B.63521 gp120D11+A244 gp120 D11+new three valent AE gp120s in GLA/SE)—new trivalent gp120s include: A A104.0 delta 11 or 7 gp120+AA107.0 delta 11 or 7gp120+AA058.1. delta 11 or 7 gp120.

Group E (placebo).

All non-placebo groups will he boosted again after periods of rest, for example 6 months.

The animals will be challenged with heterologous AE SHIV low dose rectal challenge—the AE SHIV could be either SHIV AE16 or SHIV1157 tier 2 Y173H.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. An engineered HIV-1 envelope of SEQ ID NO: 2 (B63521 Δlgp120mutC); SEQ ID NO: 4 (B.6240Δ11gp120mutC); SEQ ID NO: 6 (B.9021 gp140CmutC); SEQ ID NO: 8(B.ADAΔ11gp120mutC); or SEQ ID NO: 10 (JRFLΔ11gp120mutC).

2. A nucleic acid comprising a sequence encoding the envelope of SEQ ID NO: 2, 4, 6, 8 or 10.

3. A composition comprising any one of the envelopes of claim 1 or a combination thereof.

4. A composition comprising any one of the nucleic acids of claim 2 or a combination thereof.

5. The composition of claim 3, wherein the composition is a pharmaceutical composition comprising and adjuvant.

6. A method of inducing an immune response in a subject comprising administering to the subject a composition comprising any of the engineered envelopes of SEQ ID NOs: 2, 4, 6, 8 or 10 in an amount sufficient to effect such induction.

7. A method of inducing an immune response in a subject comprising administering to the subject the composition of claim 4 in an amount sufficient to effect such induction.

8. The method of claim 6 wherein the composition is administered as a prime.

9. The method of claim 6 wherein the composition is administered as a boost.

10. The method of claim 6 further comprising administering an adjuvant.

11. The composition of claim 4, wherein the composition is a pharmaceutical composition comprising and adjuvant.

12. A method of inducing an immune response in a subject comprising administering the composition of claim 3.

13. A method of inducing an immune response in a subject comprising administering the composition of claim 4.

14. A method of inducing an immune response in a subject comprising administering the composition of claim 5.

15. A method of inducing an immune response in a subject comprising administering the composition of claim 11.


SEQUENCE LISTING (11 sequences)

SEQ ID NO: 1 - DNA, 1557 bp, Artificial Sequence
  Description of Artificial Sequence Synthetic polynucleotide
aagcttgtcgacaccatgcgcgtgaagggcatccgcaagaactaccagcacctgtggcgctggggcaccatgctgctgggcatcctgatgatctgctccgccgtgcccgtgtggaaggaggccaccaccaccctgttctgcgcctccgacgccaaggcctacgacaccgaggtgcacaacgtgtgggccacccacgcctgcgtgcccaccgaccccaacccccaggagctggtgctggccaacgtgaccgagaacttcaacatgtggaacaacaccatggtggagcagatgcacgaggacatcatctccctgtgggaccagtccctgaagccctgcgtgaagctgacccccctgtgcgtgaccctgaactgcaccgacgtgaccaacgccaccaacatcaacgccaccaacatcaacaactcctccggcggcgtggagtccggcgagatcaagaactgctccttcaacatcaccacctccgtgcgcgacaaggtgcagaaggagtacgccctgttctacaagctggacatcgtgcccatcaccaacgagtcctccaagtaccgcctgatctcctgcaacacctccgtgctgacccaggcctgccccaaggtgtccttcgagcccatccccatccactactgcgcccccgccggcttcgccatcctgaagtgcaacaacgagaccttcaacggcaagggcccctgcatcaacgtgtccaccgtgcagtgcacccacggcatccgccccgtggtgtccacccagctgctgctgaacggctccctggccgagaaggaggtgatcatccgctccgacaacttctccgacaacgccaagaacatcatcgtgcagctgaaggagtacgtgaagatcaactgcacccgccccaacaacaacacccgcaagtccatccgcatcggccccggccagaccttctacgccaccggcgagatcatcggcaacatccgccaggcccactgcaacatctcccgctccaagtggaacgacaccctgaagcagatcgccgccaagctgggcgagcagttccgcaacaagaccatcgtgttcaacccctcctccggcggcgacctggagatcgtgacccactccttcaactgcggcggcgagttcttctactgcaacaccaccaagctgttcaactccacctggattcgcgagggcaacaacggcacctggaacggcaccatcggcctgaacgacaccgccggcaacgacaccatcatcctgccctgcaagatcaagcagatcatcaacatgtggcaggaggtgggcaaggccatgtacgccccccccatccgcggccagatccgctgctcctccaacatcaccggcctgatcctgacccgcgacggcggcaaggacgactccaacggctccgagatcctggagatcttccgccccggcggcggcgacatgcgcgacaactggcgctccgagctgtacaagtacaaggtggtgcgcatcgagcccctgggcgtggcccccacccgcgcccgcgagcgcgtggtgcagaaggagaaggagtagggatcctctaga

SEQ ID NO: 2 - protein, 509 aa, Artificial Sequence
  Description of Artificial Sequence Synthetic polypeptide
MRVKGIRKNYQHLWRWGTMLLGILMICSAVPVWKEATTTLFCASDAKAYDTEVHNVWATHACVPTDPNPQELVLANVTENFNMWNNTMVEQMHEDIISLWDQSLKPCVKLTPLCVTLNCTDVTNATNINATNINNSSGGVESGEIKNCSFNITTSVRDKVQKEYALFYKLDIVPITNESSKYRLISCNTSVLTQACPKVSFEPIPIHYCAPAGFAILKCNNETFNGKGPCINVSTVQCTHGIRPVVSTQLLLNGSLAEKEVIIRSDNFSDNAKNIIVQLKEYVKINCTRPNNNTRKSIRIGPGQTFYATGEIIGNIRQAHCNISRSKWNDTLKQIAAKLGEQFRNKTIVFNPSSGGDLEIVTHSFNCGGEFFYCNTTKLFNSTWIREGNNGTWNGTIGLNDTAGNDTIILPCKIKQIINMWQEVGKAMYAPPIRGQIRCSSNITGLILTRDGGKDDSNGSEILEIFRPGGGDMRDNWRSELYKYKVVRIEPLGVAPTRARERVVQKEKE

SEQ ID NO: 3 - DNA, 1560 bp, Artificial Sequence
  Description of Artificial Sequence Synthetic polynucleotide
aagcttgtcgacaccatgcgcgtgaagggcatccgcaagaactaccagcacctgtggcgctggggcatctggcgctggggcatcatgctgctgggcaccctgatgatctgctccgccgtgcccgtgtggaaggaggccaccaccaccctgttctgcgcctccgacgccaaggcctactcccccgagaagcacaacatctgggccacccacgcctgcgtgcccaccgaccccaacccccaggagctggtgctgggcaacgtgaccgaggacttcaacatgtggaagaacaacatggtggagcagatgcacgaggacatcatctccctgtgggaccagtccctgaagccctgcgtgaagctgacccccctgtgcgtgaccctgaactgcaccgacctgaagaactccgccaccgacaccaacggcacctccggcaccaacaaccgcaccgtggagcagggcatggagaccgagatcaagaactgctccttcaacatcaccaccggcatcggcaacaagatgcagaaggagtacgccctgttctacaagctggacgtggtgcccatcgactccaacaacaactccgacaacacctcctaccgcctgatctcctgcaacacctccgtggtgacccaggcctgccccaagacctccttcgagcccatccccatccactactgcgcccccgccggcttcgccatcctgaagtgcaacaacaagaccttctccggcaagggcccctgcaagaacgtgtccaccgtgcagtgcacccacggcatccgccccgtggtgtccacccagctgctgctgaacggctccctggccgaggaggagatcgtgatccgctccgagaacttcaccaacaacgccaagaccatcatcgtgcagctgaacgagtccgtgatcatcaactgcacccgccccaacaacaacacccgcaagtccatccgcatcggccccggccagaccttctacgccaccggcgacatcatcggcgacatccgccaggcccactgcaacctgtcctccaagtcctggaacaagaccctgcagcaggtggtgcgcaagctgcgcgagcagttcggcaacaagaccatcgccttcaaccagtcctccggcggcgaccaggagatcgtgaagcactccttcaactgcggcggcgagttcttctactgcgacaccacccagctgttcaactccacctggtcctccaacgacacctggaactccaccggcgtgcaggacaacaacatcaccctgccctgccgcatcaagcagatcatcaacatgtggcaggaggtgggcaaggccatgtacgccccccccatccagggcctgatctcctgctcctccaacatcaccggcctgctgctgacccgcgacggcggcaccaacaacaccaacgccaccgagatcttccgccccggcggcggcgacatgcgcgacaactggcgctccgagctgtacaagtacaaggtggtgaagatcgagcccctgggcatcgcccccaccaaggccaagcgccgcgtggtgcagcgcgagaagcgctagggatcctctaga

SEQ ID NO: 4 - protein, 510 aa, Artificial Sequence
  Description of Artificial Sequence Synthetic polypeptide
MRVKGIRKNYQHLWRWGIWRWGIMLLGTLMICSAVPVWKEATTTLFCASDAKAYSPEKHNIWATHACVPTDPNPQELVLGNVTEDFNMWKNNMVEQMHEDIISLWDQSLKPCVKLTPLCVTLNCTDLKNSATDTNGTSGTNNRTVEQGMETEIKNCSFNITTGIGNKMQKEYALFYKLDVVPIDSNNNSDNTSYRLISCNTSVVTQACPKTSFEPIPIHYCAPAGFAILKCNNKTFSGKGPCKNVSTVQCTHGIRPVVSTQLLLNGSLAEEEIVIRSENFTNNAKTIIVQLNESVIINCTRPNNNTRKSIRIGPGQTFYATGDIIGDIRQAHCNLSSKSWNKTLQQVVRKLREQFGNKTIAFNQSSGGDQEIVKHSFNCGGEFFYCDTTQLFNSTWSSNDTWNSTGVQDNNITLPCRIKQIINMWQEVGKAMYAPPIQGLISCSSNITGLLLTRDGGTNNTNATEIFRPGGGDMRDNWRSELYKYKVVKIEPLGIAPTKAKRRVVQREKR

SEQ ID NO: 5 - DNA, 2088 bp, Artificial Sequence
  Description of Artificial Sequence Synthetic polynucleotide
gtcgacaccatgcgcgtgaagggcatccgcaagaactgccagcagcacctgtggcgctggggcaccatgctgctgggcatcctgatgatctgctccgccgccgagaacctgtgggtgaccgtgtactacggcgtgcccgtgtggaaggaggccaccaccaccctgttctgcgcctccgacgccaaggcctacgacaccgaggtgcacaacgtgtgggccacccacgcctgcgtgcccaccgaccccaacccccaggagatggtgctggagaacgtgaccgagtacttcaacatgtggaagaacaacatggtggagcagatgcacgaggacatcatctccctgtgggaccagtccctgaagccctgcgtgaagctgacccccctgtgcgtgaccctgacctgcaccgactacgagtggaactgcaccggcatccgcaactccatctgcaagtacaacaacatgaccaacaactcctcctccggcaactacaccggctgggagcgcggcgagatcaagaactgctccttcaactccaccatctccggcatccgcgacaaggtgcgcaaggagtacgccctgctgtacaagatcgacctggtgtccatcgacggctccaacacctcctaccgcatgatctcctgcaacacctccgtgatcacccagtcctgccccaagatctccttcgagcccatccccctgcactactgcacccccgccggcttcgccctgctgaagtgcaacgacaagaagttcaacggcaccggcctgtgccacaacgtgtccaccgtgcagtgcacccacggcatcaagcccgtggtgtccacccagctgctgctgaacggctccctggccgaggaggaggtggtgatccgctccaagaacttcaccgacaacgccaagatcatcatcgtgcagctgaacgagaccgtggagatcaactgcacccgccccaacaacaacacccgcaagtccatccgcatcggccccggccagaccttctacgccaccggcgagatcatcggcgacatccgccgcgcccactgcaacatctcccgcgagaagtggaacaccaccctgcaccgcatcgccaccaagctgcgcgagcagtacaacaagaccatcgtgttcaaccagtcctccggcggcgaccccgagatcgtgatgcactccgtgaactgcggcggcgagttcttctactgcaacacctccaagctgttcaactccacctggaactccaccggcggctccatctccgaggactccgagaacatcaccctgccctgccgcatcaagcagatcgtgaacatgtggcaggaggtgggcaaggccatgtacgccccccccatccgcggccagatccgctgctcctccaacatcaccggcctgctgctgacccgcgacggcggcatcaaccagtccatctccgagaccttccgccccggcggcggcgacatgcgcgacaactggcgctccgagctgtacaagtacaaggtggtgaagatcgagcccctgggcatcgcccccaccaaggcccgcgagcgcgtggtgcagcgcgagaaggaggccgtgggcatcggcgccgtgttcctgggcttcctgggcgccgccggctccaccatgggcgccgcctccctgaccctgaccgtgcaggcccgcctgctgctgtccggcatcgtgcagcagcagaacaacctgctgcgcgccatcgaggcccagcagcacatgctgcagctgaccgtgtggggcatcaagcagctgcaggcccgcgtgctggccctggagcgctacctgcgcgaccagcagctgatgggcatctggggctgctccggcaagctgatctgcaccaccgccgtgccctggaacgcctcctggtccaacaagtccctgaacgacatctggaacaacatgacctggatgcagtgggagcgcgagatcgacaactacaccggcctgatctactccctgctggaggagtcccagaaccagcaggagaagaacgagcaggacctgctggccctggacaagtgggccaacctgtggacctggttcgacatctccaactggctgtggtacatcaagtagggatcc

SEQ ID NO: 6 - protein, 690 aa, Artificial Sequence
  Description of Artificial Sequence Synthetic polypeptide
MRVKGIRKNCQQHLWRWGTMLLGILMICSAAENLWVTVYYGVPVWKEATTTLFCASDAKAYDTEVHNVWATHACVPTDPNPQEMVLENVTEYFNMWKNNMVEQMHEDIISLWDQSLKPCVKLTPLCVTLTCTDYEWNCTGIRNSICKYNNMTNNSSSGNYTGWERGEIKNCSFNSTISGIRDKVRKEYALLYKIDLVSIDGSNTSYRMISCNTSVITQSCPKISFEPIPLHYCTPAGFALLKCNDKKFNGTGLCHNVSTVQCTHGIKPVVSTQLLLNGSLAEEEVVIRSKNFTDNAKIIIVQLNETVEINCTRPNNNTRKSIRIGPGQTFYATGEIIGDIRRAHCNISREKWNTTLHRIATKLREQYNKTIVFNQSSGGDPEIVMHSVNCGGEFFYCNTSKLFNSTWNSTGGSISEDSENITLPCRIKQIVNMWQEVGKAMYAPPIRGQIRCSSNITGLLLTRDGGINQSISETFRPGGGDMRDNWRSELYKYKVVKIEPLGIAPTKARERVVQREKEAVGIGAVFLGFLGAAGSTMGAASLTLTVQARLLLSGIVQQQNNLLRAIEAQQHMLQLTVWGIKQLQARVLALERYLRDQQLMGIWGCSGKLICTTAVPWNASWSNKSLNDIWNNMTWMQWEREIDNYTGLIYSLLEESQNQQEKNEQDLLALDKWANLWTWFDISNWLWYIK

SEQ ID NO: 7 - DNA, 1518 bp, Artificial Sequence
  Description of Artificial Sequence Synthetic polynucleotide
aagcttgtcgacaccatgcgcgtgatgggcatccagcgcaactacccccagtggtggatctggtccatgctgggcttctggatgctgatgatctgcaacggcgtgcccgtgtggaaggaggccaccaccaccctgttctgcgcctccgacgccaaggcctacgacaccgaggtgcacaacgtgtgggccacccacgcctgcgtgcccaccgaccccaacccccaggaggtggtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccgacctgcgcaacgtgaccaacatcaacaactcctccgagggcatgcgcggcgagatcaagaactgctccttcaacatcaccacctccatccgcgacaaggtgaagaaggactacgccctgttctaccgcctggacgtggtgcccatcgacaacgacaacacctcctaccgcctgatcaactgcaacacctccaccatcacccaggcctgccccaaggtgtccttcgagcccatccccatccactactgcacccccgccggcttcgccatcctgaagtgcaaggacaagaagttcaacggcaccggcccctgcaagaacgtgtccaccgtgcagtgcacccacggcatccgccccgtggtgtccacccagctgctgctgaacggctccctggccgaggaggaggtggtgatccgctcctccaacttcaccgacaacgccaagaacatcatcgtgcagctgaaggagtccgtggagatcaactgcacccgccccaacaacaacacccgcaagtccatccacatcggcccccagaccgccttctacaccaccggcgagatcatcggcgacatccgccaggcccactgcaacatctcccgcaccaagtggaacaacaccctgaaccagatcgccaccaagctgaaggagcagttcggcaacaacaagaccatcgtgttcaaccagtcctccggcggcgaccccgagatcgtgatgcactccttcaactgcggcggcgagttcttctactgcaactccacccagctgttcaactccacctggaacttcaacggcacctggaacctgacccagtccaacggcaccgagggcaacgacaccatcaccctgccctgccgcatcaagcagatcatcaacatgtggcaggaggtgggcaaggccatgtacgccccccccatccgcggccagatccgctgctcctccaacatcaccggcctgatcctgacccgcgacggcggcaccaactcctccggctccgagatcttccgccccggcggcggcgacatgcgcgacaactggcgctccgagctgtacaagtacaaggtggtgaagatcgagcccctgggcgtggcccccaccaaggccaaggagcgcgtggtgcagcgcgagaaggagtagggatcctctaga

SEQ ID NO: 8 - protein, 496 aa, Artificial Sequence
  Description of Artificial Sequence Synthetic polypeptide
MRVMGIQRNYPQWWIWSMLGFWMLMICNGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPQTAFYTTGEIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGTNSSGSEIFRPGGGDMRDNWRSELYKYKVVKIEPLGVAPTKAKERVVQREKE

SEQ ID NO: 9 - DNA, 1503 bp, Artificial Sequence
  Description of Artificial Sequence Synthetic polynucleotide
aagcttgtcgacaccatgcgcgtgatgggcatccagcgcaactacccccagtggtggatctggtccatgctgggcttctggatgctgatgatctgcaacggcgtgcccgtgtggaaggaggccaccaccaccctgttctgcgcctccgacgccaaggcctacgacaccgaggtgcacaacgtgtgggccacccacgcctgcgtgcccaccgaccccaacccccaggaggtggtgctggagaacgtgaccgagcacttcaacatgtggaagaacaacatggtggagcagatgcaggaggacatcatctccctgtgggaccagtccctgaagccctgcgtgaagctgacccccctgtgcgtgaccctgaactgcaaggacgtgaacgccaccaacaccaccaacgactccgagggcaccatggagcgcggcgagatcaagaactgctccttcaacatcaccacctccatccgcgacgaggtgcagaaggagtacgccctgttctacaagctggacgtggtgcccatcgacaacaacaacacctcctaccgcctgatctcctgcgacacctccgtgatcacccaggcctgccccaagatctccttcgagcccatccccatccactactgcgcccccgccggcttcgccatcctgaagtgcaacgacaagaccttcaacggcaagggcccctgcaagaacgtgtccaccgtgcagtgcacccacggcatccgccccgtggtgtccacccagctgctgctgaacggctccctggccgaggaggaggtggtgatccgctccgacaacttcaccaacaacgccaagaccatcatcgtgcagctgaaggagtccgtggagatcaactgcacccgccccaacaacaacacccgcaagtccatccacatcggccccggccagaccttctacaccaccggcgagatcatcggcgacatccgccaggcccactgcaacatctcccgcgccaagtggaacgacaccctgaagcagatcgtgatcaagctgcgcgagcagttcgagaacaagaccatcgtgttcaaccactcctccggcggcgaccccgagatcgtgatgcactccttcaactgcggcggcgagttcttctactgcaactccacccagctgttcaactccacctggaacaacaacaccgagggctccaacaacaccgagggcaacaccatcaccctgccctgccgcatcaagcagatcatcaacatgtggcaggaggtgggcaaggccatgtacgccccccccatccgcggccagatccgctgctcctccaacatcaccggcctgctgctgacccgcgacggcggcatcaacgagaacggcaccgagatcttccgccccggcggcggcgacatgcgcgacaactggcgctccgagctgtacaagtacaaggtggtgaagatcgagcccctgggcgtggcccccaccaaggccaaggagcgcgtggtgcagcgcgagaaggagtagggatcctctaga

SEQ ID NO: 10 - protein, 491 aa, Artificial Sequence
  Description of Artificial Sequence Synthetic polypeptide
MRVMGIQRNYPQWWIWSMLGFWMLMICNGVPVWKEATTTLFCASDAKAYDTEVHNVWATHACVPTDPNPQEVVLENVTEHFNMWKNNMVEQMQEDIISLWDQSLKPCVKLTPLCVTLNCKDVNATNTTNDSEGTMERGEIKNCSFNITTSIRDEVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGQTFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMRDNWRSELYKYKVVKIEPLGVAPTKAKERVVQREKE

SEQ ID NO: 11 - protein, 33 aa, Human immunodeficiency virus-1
TRPNNNTRKSIRIGPGQTFYATGDIIGNIRQAH